(19) United States
(12) Patent Application Publication
(10) Pub. No.: US 2007/0264638 A1
(43) Pub. Date: Nov. 15, 2007

(54) Identification of ginseng and its imposters using genetic variations

(75) Inventors: Fan Zhang, Chesterfield, MO (US); Keming Song, Frontenac, MO (US); Nathan Zenser, Rock Hill, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Sigma-Aldrich Co.

(21) Appl. No.: 11/431,375

(22) Filed: May 10, 2006

(51) Int. Cl. C12Q 1/68

(52) U.S. Cl. 435/6

(57) ABSTRACT

This invention provides methods, compounds, and kits for identifying ginseng (Panax) species and for distinguishing between ginseng and imposter species based upon molecular variations. In particular, amplification primers are provided that amplify polymorphic regions of nucleic acid sequences to generate distinct amplification profiles in the different plant species. A plant may be identified by comparing its amplification profile to known amplification profiles of Panax and imposter species.

### FIELD OF THE INVENTION

The present invention provides compositions, methods, and kits for distinguishing between ginseng (Panax) and imposter species and also for distinguishing among the different species of Panax.

### BACKGROUND OF THE INVENTION

Ginseng is a slow-growing perennial plant with fleshy roots that has been prized for many years for its medicinal properties. Ginseng products are popularly referred to as “tonics” because they purportedly increase the body's resistance to stress and enhance its vitality. Among the many reported benefits of ginseng include its ability to boost physical and mental endurance, increase disease resistance, enhance sexual desire, strengthen the cardiovascular and nervous systems, slow the aging process, and prevent memory loss.

Ginseng species comprise the genus Panax, whose name is derived from the Greek Panakos (a panacea), in reference to ginseng's ability to “cure all.” While Korean ginseng (Panax ginseng) is widely used, the American ginseng (Panax quinquefolius) is considered superior for certain application (e.g., gastrointestinal problems). As a consequence, American ginseng is generally more expensive than Korean ginseng. Ginsenosides are the complex carbohydrate compounds considered to be the active ingredients in ginseng. Up to 29 different ginsenosides have been identified (Hon et al. 2003, Acta Pharmacol. 24(9): 841-846).

Because of ginseng's popularity, other plants have been sold or passed off as ginseng. Currently, the most common imposter plant is Eleutherococcus senticosis, or Siberian ginseng. Although E. senticosis is morphologically similar to Panax, it lacks the ginsenosides found in Panax. It may contain other biologically active substances, however, and is marketed for certain indications.

Various methods to identify or authenticate Panax species have been developed. Methods that rely on morphological or anatomical features can only be used with intact plant material, and thus have limited utility. Chemical and chromatographic techniques that identify plants on the basis of the active ingredients are not only labor intensive, but tend to be unreliable. They are not dependable because the chemical composition of a plant can vary from grower to grower, crop to crop, and is affected by processing steps and storage conditions. Methods that exploit genetic differences, however, show the most promise because the genetic material is quite stable. Hybridization techniques, such as RFLP or PCR-RLFP, have been developed in which Panax species can be distinguished from each other (U.S. Pat. No. 5,876,977; U.S. Patent Application Publication No. 2002/0146705; Um et al, 2001, Biol. Pharm. Bull. 24: 1210-1213.). Methods using the direct amplification of polymorphic regions of DNA have permitted different species of Panax to be distinguished (U.S. Pat. No. 6,803,215; Ha et al., 2001, Planta Med. 67: 587-589).

At present, no methods exit for distinguishing between an authentic Panax species and an imposter species. Thus, there is a need for a quick, reliable, reproducible method that will distinguish between Panax and imposter species and will also distinguish among the different species of Panax.

### BRIEF SUMMARY OF THE INVENTION

The present invention provides oligonucleotide primers, methods, and kits to distinguish between Panax and imposter species, as well as identify the different species of Panax.

One aspect of the invention is the provision of amplification primers that amplify a polymorphic region of a nucleic acid sequence and generate a distinct profile of amplified products in each species. The amplified products may vary in number, size, and/or abundance.

Another aspect of the invention provides methods for distinguishing between Panax and imposter species and for distinguishing among the different species of Panax. The method comprises amplifying a polymorphic region of a nucleic acid sequence from a plant using at least one pair of the amplification primers, and identifying the plant on the basis of its amplification profile in comparison to the known amplification profiles of Panax and imposter species.

An additional aspect of the invention provides kits for distinguishing between Panax and imposter species and for distinguishing among the different species of Panax. A kit comprises the amplification primers of the invention, instructions for use, and known amplification profiles of Panax and imposter species. A kit may further comprise solutions of reaction buffer, dNTP mix, divalent cation, and Taq DNA polymerase.

Other aspects and features of the invention are described in more detail herein.

### BRIEF DESCRIPTION OF THE DRAWINGS

FIG. 1 depicts an image of a gel showing the unique set of different sized PCR products in each species. Presented is a reverse image of an ethidium bromide stained 1.2% agarose gel. Products amplified with the multiplex primer set 1 (SEQ ID NOs:1-6) are shown on the left, and products amplified with the multiplex primer set 2 (SEQ ID NOs:4-10) are shown on the right. The two outer lanes display DNA molecular size standards, with fragments ranging from 50 bp to 2,000 bp. AG stands for American ginseng (Panax quinquefolius). KG stands for Korean ginseng (P. ginseng), TG stands for Tianqi ginseng (P. notoginseng), and SG stands for Siberian ginseng (Eleutherococcus senticosus).

### DETAILED DESCRIPTION OF THE PREFERRED EMBODIMENTS

Specific amplification primers have been discovered, as detailed in the examples, that amplify a polymorphic region of a nucleic acid sequence and may generate a unique profile of amplified products that vary in number, size, and/or abundance in each species. These primers may be used to determine whether a plant is a true ginseng (Panax) species or an imposter, and they may be used to distinguish among the different species of Panax.

I. Amplification Primers

One aspect of this invention provides amplification primers that generate different profiles of products in the different species, such that the different Panax or imposter species may be distinguished. An amplification primer is a short strand of nucleotides that anneals to a complementary nucleic acid sequence, and serves as a starting point for replication. During an amplification reaction, the region between the annealing sites of two primers is amplified many fold. The amplified region may vary in length in the different species due to insertions or deletions between the annealing sites. The amount (abundance) of amplified product may vary due to mismatches between the primer and the annealing site, which may lead to inefficient priming of the reaction such that less product is generated in some species. Lastly, a region may not be amplified in one species because it lacks one or both of the primer annealing sites. Thus, the profile of amplified products in each species may vary in number, size, and/or abundance.

Polymorphic regions may be found in DNA or RNA sequences. In a preferred embodiment, the nucleic acid sequence to be amplified is DNA. The DNA may be nuclear DNA, mitochondrial DNA, or chloroplast DNA. DNA polymorphisms may be found within protein-coding genes, introns, RNA-coding genes, non-coding regions of DNA, transcribed spacer regions, or non-transcribed spacer regions. In one embodiment, the polymorphic region to be amplified is within in a protein-coding gene. In another embodiment, the polymorphic region to be amplified is within the transcribed region of a ribosomal RNA-coding gene.

The size of the amplification products can and will vary without departing from the scope of the invention. In general, the sizes of the amplification products are typically such that they are amenable to PCR amplification. For products generated by traditional PCR, the amplification products generally are of sizes that may be quickly and easily characterized after the PCR reaction. For example, the products preferably are of sizes that may be resolved readily by gel electrophoresis. In one embodiment, the amplified products may range from about 50 bp to 5,000 bp in length. In another embodiment, the amplified products may range from about 100 bp to about 3,000 bp in length. In a preferred embodiment, the amplified products may range from about 300 bp to about 1,200 bp in length.

The following oligonucleotide primers are provided by this invention: SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. The invention also encompasses a primer whose sequence differs from the sequence of any of the primers identified herein, but which may still hybridize to and amplify the same polymorphic region of DNA. In one embodiment, the amplification primer has a nucleotide sequence that is at least 75% identical to the sequence of either SEQ ID NOs:1, 2, 3, 4, 5, 6, 7, 8, 9, or 10. In another embodiment, the amplification primer has a nucleotide sequence that is at least 80, 85, or 90% identical to the sequence of either SEQ ID NOs: 1, 2, 3, 4, 5, 6, 7, 8, 9, or 10. In still another embodiment, the amplification primer has a nucleotide sequence that is at least 91, 92, or 93% identical to the sequence of either SEQ ID NOs: 1, 2, 3, 4, 5, 6, 7, 8, 9, or 10. In an alternative embodiment, the amplification primer has a nucleotide sequence that is at least 94, 95, or 96% identical to the sequence of either SEQ ID NOs: 1, 2, 3, 4, 5, 6, 7, 8, 9, or 10. In yet another embodiment, the amplification primer has a nucleotide sequence that is at least 97, 98, or 99% identical to the sequence of either SEQ ID NOs:1, 2, 3, 4, 5, 6, 7, 8, 9, or 10.

Conventional algorithms may be used to determine the percent of nucleotide sequence identity between different nucleic acid sequences. In particular, the percent of identity between two nucleic acid sequences is determined using the algorithm of Karlin and Altschul (Proc. Natl. Acad. Sci. USA 87:2264-2268,1993). Such an algorithm is incorporated into the NBLAST and XBLAST programs of Altschul et al. (J. Mol. Biol. 215:403-410, 1990). BLAST nucleotide searches may be performed with the NBLAST program to obtain nucleotide sequences homologous to the primers of the invention. See http://www.ncbi.nlm.nih.gov for more details.

Hybridization of an oligonucleotide probe to a nucleic acid sample is typically performed under stringent conditions. Nucleic acid duplex or hybrid stability is expressed as the melting temperature or Tₘ, which is the temperature at which a probe dissociates from a target DNA. The Tₘ for perfectly matched hybrids may be estimated using the following equation: Tₘ=81.5−16.6(log₁₀[Na⁺])+0.41(% G+C)−(600/N), where N=length of the probe in nucleotides. This equation generally predicts reasonably well the Tₘ for oligonucleotides as long as about 70 nucleotides and as short as about 14 nucleotides. The Tₘ for imperfectly matched hybrids may be estimated by subtracting about 1° C. from the calculated Tₘ for each 1% of mismatch. For example, if a sequence having greater than 95% identity with the primer is sought, the final temperature is decreased by approximately 5° C. In practice, the change in Tₘ may be between 0.5-1.5° C. per 1% mismatch. When using oligonucleotides as probes, the selected hybridization conditions are typically stringent enough to guarantee specificity, but are flexible enough to allow formation of stable hybrids at an acceptable rate. Typically, hybridizations with oligonucleotides are performed at about 5-10° C. below the Tₘ of the perfect hybrid. Thus, oligonucleotide hybridizations are typically performed in 5×SSC/5× Denhardt's solution/1.0% SDS, and the washes are done in 2×SSC/0.1% SDS at the estimated temperature. The parameters of salt concentration and temperature may be varied during the washes to achieve the optimal level of hybridization between the probe and the subject nucleotide sequence. Additional guidance regarding such conditions is readily available in the art, for example, by Sambrook et al., 1989, Molecular Cloning, A Laboratory Manual, Cold Spring Harbor Press, N.Y.; and Ausubel et al., (eds.), 1995, Current Protocols in Molecular Biology, (John Wiley & Sons, N.Y.) at Unit 2.10.

The invention also includes a primer that is essentially identical in sequence to any of those disclosed herein, but which further comprises one or more restriction endonuclease recognition sequences added to either end of the primer. Similarly, the invention encompasses a primer that is essentially identical in sequence to any of those disclosed herein, but which further comprises from about 1 to about 20 nucleotides added to either end of the primer.

The invention encompasses amplification primers with standard nucleobases (adenosine, guanosine, cytosine, and thymidine), as well as primers nonstandard nucleobases. Non-limiting examples of nonstandard nucleobases include inosine, xanthosine, isoguanosine, isocytidine, diaminopyrimidine, and deoxyuridine. An amplification primer may contain standard and/or nonstandard nucleotides linked by phosphothioate linkages as well as phosphodiester linkages. The invention also encompasses amplification primers with modified or derivatized nucleotides. Non-limiting examples of modifications on the ribose or base moieties of nucleotide molecules include the addition (or removal) of acetyl groups, amino groups, carboxyl groups, carboxymethyl groups, hydroxyl groups, methyl groups, phosphoryl groups, and thiol groups. In particular, 2′-O-methyl and LNA nucleotides are included. Suitable examples of derivatized nucleotides include those with covalently attached dyes, such as fluorescein- or rhodamine-based fluorescent dyes or quenching dyes, or other molecules, such as biotin or digoxygenin.

In a preferred embodiment, SEQ ID NOs:1-10 comprise standard, non-modified, non-derivatized deoxynucleotides linked by phosphodiester bonds that amplify DNA products ranging in size from about 300 bp to about 1200 bp.

II. Methods for Distinguishing Between Panax and Imposter Species and for Distinguishing Among the Different Species of Panax

Another aspect of the invention includes methods for determining whether a plant is a Panax species or an imposter species and for distinguishing among the different species of Panax. The use of this invention allows for the authentication of medicinal plants, as well as the quality control and standardization of herbal medicines.

(a) Panax Species

The present invention provides a method for distinguishing among the different species of Panax on the basis of the different amplification profiles produced by the amplification primers (as shown in Example 2). Thus, this invention provides a means to identify the different species of Panax. The method may be used to distinguish among those species of Panax used most frequently for medicinal purposes, i.e., P. quinquefolius (American ginseng), P. ginseng (Korean ginseng), and P. notoginseng (Tianqi ginseng). The method may be utilized to identify other species of Panax, such as, P. bipinnatifidus (pearl ginseng), P. japonicus (Japanese ginseng), P. pseudoginseng (Himalayan ginseng), P. trifolius (dwarf ginseng), P. vietnamensis (Vietnamese ginseng), P. wangianus (Assamese ginseng), and P. zingiberensis (ginger ginseng)

(b) Imposter Species

The invention provides a method for distinguishing between Panax species and imposter species on the basis of the different amplification profiles produced by the amplification primers. As demonstrated in Example 2, the method of the invention distinguishes between Panax species and the imposter, Eleutherococcus senticosis. The method of the invention may also allow discrimination between Panax species and other imposter species. Non-limiting examples of imposter species include Angelica sinensis (female ginseng), Aralia spp. (wild ginseng), Campanumoea pilosula (bastard ginseng), Caulophyllum thalictroides (blue or yellow ginseng), Eleutherococcus gracilstylus (prickly ginseng), Eurycoma longifolia (Malaysian ginseng), Gynostemma pentaphyllum (Southern or blue ginseng), Lepidium meyenii (Peruvian ginseng), Oplopanax horridus (Pacific ginseng), Pfaffia paniculata (Brazilian ginseng), Pseudostellaria heterophylla (prince or lesser ginseng), Rumex hymenosepalus (wild red desert ginseng), Trichopus zeylanicus (Indian ginseng), and Withania somnifera (Indian or Ayurvedic ginseng).

(c) Plant Material

Plant materials of all kinds are appropriate sources of nucleic acids for practice of this invention. The plant material may be from cultivated or wild stocks. The plant material may be fresh, dried, frozen, or stored in an aqueous solution. The plant material may be derived from roots, root prongs, root fibers, stems, leaves, leaflets, flowers, or seeds. The plant material may be processed with heat to produce red ginseng (as opposed to white ginseng which is not heat treated). The plant material may be whole, shredded, sliced, diced, crushed, and pulverized. The plant material may be further processed and be in the form of capsules, tablets, powders, teas, aqueous extracts, alcohol extracts, candy, or gum.

Nucleic acids may be isolated from plant materials using methods well known in the art. In a preferred embodiment, DNA is extracted from roots using standard procedures. The DNA may be highly purified or not very well purified, which means that other plant-based compounds may be present, as long as they do not interfere with the amplification reaction.

(d) Amplification of Polymorphic Regions

The present invention provides sets of primers that amplify polymorphic regions of DNA and yield distinct amplification profiles in the different species. In one embodiment, multiplex PCR is performed using SEQ ID NOs:1-6. In another embodiment, multiplex PCR is performed using SEQ ID NOs:4-10.

The amplification reaction may be performed using traditional PCR or real time PCR. In one embodiment, traditional PCR is performed with the above-mentioned primer pairs using methods well known in the art. The PCR products may be resolved on 1-2% agarose gels or 8-15% polyacrylamide gels and visualized by staining with fluorescent dyes, such as ethidium bromide or SYTOX Green. In another embodiment, real time PCR is performed using the above-mentioned primers that have been derivatized with a fluorescent dye (and a quencher dye). Fluorescent dyes with different emission spectra may be attached to the different primers for multiplex PCR. The amplified products may be quantified using methods known to a skilled artisan.

(e) Identification Based Upon Distinct Amplification Profiles

The primers of the invention typically amplify polymorphic regions of DNA, such that they produce products that differ in number, size, and/or abundance in the different species. For example, amplification with SEQ ID NOs: 4-10 produces distinct amplification profiles in each species (see Example 2). Thus, each species essentially has a unique fingerprint. These fingerprints may be used to reveal the identity of a test plant, by comparing the amplification profile of the test plant with known amplification profiles of Panax and imposter species. Furthermore, it is also possible that the amplification profile a test plant may be a blend of profiles, indicating that it is a mixture of Panax species or a mixture of Panax and imposter species.

In a preferred embodiment, multiplex PCR is performed using SEQ ID Nos:1-6 to amplify polymorphic regions of DNA from a test plant. In another preferred embodiment, multiplex PCR is performed using SEQ ID Nos:4-10 to amplify polymorphic regions of DNA from a test plant. For both of these embodiments, the products are resolved by agarose gel electrophoresis and the identity of the plant is determined by comparing its amplification profile against the known amplification profiles of Panax and imposter species.

III. Kits for Distinguishing Between Panax and Imposter Species and for Distinguishing Among the Different Species of Panax

Another aspect of the invention encompasses kits for determining whether a plant is a Panax species or an imposter species and for distinguishing among the species of Panax. In general, a kit comprises amplification primers of the invention, instructions for use, and known amplification profiles of Panax and imposter species. In one embodiment, the amplification primers comprise SEQ ID NOs:1-6. In another embodiment, the amplification primers comprise SEQ ID NOs:4-10. The kit may further comprise solutions of reaction buffer, dNTPs, MgCl₂, and Taq DNA polymerase for the amplification reaction.

### DEFINITIONS

The term “amplification primer” used herein refers to an oligonucleotide (a short strand of nucleotides) that anneals or hybridizes to a complementary nucleic acid sequence, whereby the region between two primer annealing sites is amplified many fold.

The term “amplification profile” used herein refers to the unique set of products amplified in each plant species. The amplified products may vary in number, size, and/or abundance. Abundance is a reflection of the efficiency of amplification, or the amount of product generated.

As used herein, the terms “imposter” or “imposter species” refer to a plant that may be called ginseng or may be sold as ginseng, but is not a true ginseng (i.e., is not a Panax species). An imposter generally lacks the active substances found in Panax, but it may contain other active ingredients.

The term “polymorphic region” refers to a stretch of a nucleic acid sequence that is relatively variable between species. Variations within a stretch of DNA may be due to differences in length between two reference points.

As various changes could be made in the above compounds, methods, and products without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Design and Characterization of PCR Primer Pairs

There are currently three commercially relevant ginseng species: Panax quinquefolius (American ginseng), Panax ginseng (Korean ginseng) and Panax notoginseng (Tianqi ginseng). A common imposter mistaken for Panax is Eleutherococcus senticosus (Siberian ginseng). PCR amplification primers that would generate a unique set of DNA fragments in each species were identified using the following strategy. First, sets of cDNA sequences for P. ginseng from GenBank were aligned to identify unique sequences and primer sets were designed using the vector NTI program. Second, specific regions of the genome of each species were sequenced and gene-specific primers were designed using the vector NTI program. The primers were synthesized by Sigma-Genosys.

The primers were screened by PCR to identify primer sets that produced polymorphic DNA fragments in the different species. PCR was performed using JumpStart REDTaq ReadyMix (P0982, Sigma-Aldrich, St. Louis, Mo.) containing 3 mM MgCl₂ and 0.75 μM of each primer; the cycling parameters were 94° C. for 5 min, 25 cycles of 94° C. for 15 sec, 60° C. for 45 sec, and 72° C. for 1 min, followed by 72° C. for 7 min and 4° C. indefinitely. Aliquots of the reactions were resolved on 1.2% agarose gels.

In some cases, non-polymorphic DNA fragments from the first PCR run were isolated and sequenced to identify polymorphic sites at the nucleotide level. So far, about 10% to 15% of the tested primer sets have generated polymorphic fragments among the different species. In general, DNA sequence alignments that revealed polymorphic regions in any given gene among the different species were used to design species-specific primers, which were then tested. Combinations of primer pairs were selected for multiplex PCR to distinguish among the different ginseng species and distinguish the real ginseng species from imposter species by generating different banding patterns in each species.

### Example 2

### Multiplex PCR

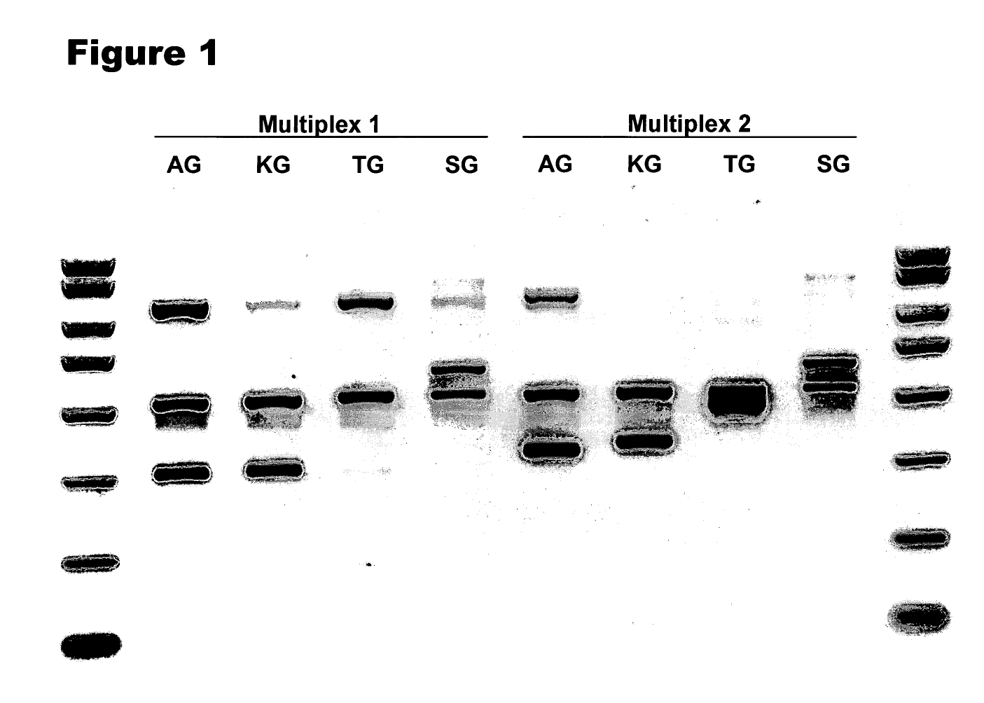

Two sets of multiplex primers (Tables 1 and 2) were selected and tested in P. quinquefolius (American ginseng; AG), P. ginseng (Korean ginseng; KG), P. notoginseng (Tianqi ginseng; TG), and the imposter Eleutherococcus senticosus (Siberian ginseng; SG). The PCR conditions were as described above, except the ITS primers were used at 0.375 μM. Unique sets of products of different sizes were amplified in each species as shown in FIG. 1. These data reveal that the amplification primers described in this invention can be used to produce unique DNA fragments that may be used to distinguish the true ginseng species from the imposter species and from each other.

TABLE 1

Ginseng Primer Set 1.

SEQ

| | | |
|---|---|---|
| Primer | Accession | ID |

| | | | |
|---|---|---|---|
| name | Sequence | No. | NO |

| | | | |
|---|---|---|---|
| cDNA24-F | GCCACTATATTTCCCCTATTTCTCTCCTC | CN848658 | 1 |
| | CC | | |
| cDNA24-R | CAGCCATTTAGCCTTCGACTTATGTGC | CN848658 | 2 |
| TG24-R | TTTGGTTTTGCCTTCTTCAGGTAATA | — | 3 |
| GITS-F | TGAACCTGCGGAAGGATCATTGTC | — | 4 |
| GITS-R | ACTCGCATTTGGGCCAACCG | — | 5 |
| SGITS-R | CGAGGACGGCACAACAGGGTCACAT | — | 6 |

TABLE 2

Ginseng Primer Set 2.

SEQ

| | | |
|---|---|---|
| Primer | Accession | ID |

| | | | |
|---|---|---|---|
| name | Sequence | No. | NO |

| | | | |
|---|---|---|---|
| cDNA21-F | TCAGTACTGGGATTACCTGAAATGCTCTT | CN848662 | 7 |
| | GG | | |
| cDNA21-R | CGAGCCATTTGGATCATGAAAGGAAG | CN848662 | 8 |
| cDNA41-F | ACTGGCTAGCTAGCTAGCTCGATCGCTC | CN847145 | 9 |
| cDNA41-R | GGCTCTCCCTTTGTTGCATTGAAGT | CN847145 | 10 |
| GITS-F | TGAACCTGCGGAAGGATCATTGTC | — | 4 |
| GITS-R | ACTCGCATTTGGGCCAACCG | — | 5 |
| SGITS-R | CGAGGACGGCACAACAGGGTCACAT | — | 6 |

What is claimed is:

1. A method for determining whether a plant is a Panax species or an imposter species, the method comprising:
a. amplifying a polymorphic region of a nucleic acid sequence from the plant using at least one pair of amplification primers, whereby the primers generate a specific amplification profile of products in each species; and
b. identifying the plant by comparing its amplification profile against known amplification profiles of Panax and imposter species.

2. The method of claim 1, wherein the amplification primers comprise SEQ ID NOs:1-6.

3. The method of claim 2, wherein the primers are at least seventy-five percent identical in nucleotide sequence to SEQ ID NOs:1-6.

4. The method of claim 1, wherein the amplification primers comprise SEQ ID NOs:4-10.

5. The method of claim 4, wherein the primers are at least seventy-five percent identical in nucleotide sequence to SEQ ID NOs:4-10.

6. The method of claim 1, wherein the nucleotides comprising the primers are selected from the group consisting of standard, nonstandard, modified, and derivatized nucleotides.

7. The method of claim 1, wherein the Panax species is selected from the group consisting of P. quinquefolius, P. ginseng, P. notoginseng, P. bipinnatifidus, P. japonicus, P. pseudoginseng, P. trifolius, P. vietnamensis, P. wangianus, and P. zingiberensis.

8. The method of claim 1, wherein the imposter species is selected from the group consisting of Eleutherococcus senticosis, Angelica sinensis, Aralia spp., Campanumoea pilosula, Caulophyllum thalictroides, Eleutherococcus gracilstylus, Eurycoma longifolia, Gynostemma pentaphyllum, Lepidium meyenii, Oplopanax horridus, Pfaffia paniculata, Pseudostellaria heterophylla, Rumex hymenosepalus, Trichopus zeylanicus, and Withania somnifera.

9. The method of claim 1, wherein the Panax species is selected from the group consisting of P. quinquefolius, P. ginseng, and P. notoginseng, and the imposter species is Eleutherococcus senticosis.

10. The method of claim 1, wherein the plant is a material selected from the group consisting of roots, root prongs, root fibers, stems, leaves, leaflets, flowers, and seeds.

11. The method of claim 10, wherein the plant material is selected from the group consisting of whole, shredded, sliced, diced, crushed, pulverized, and heated material.

12. The method of claim 1, wherein the plant is a processed plant material selected from the group consisting of capsules, tablets, powders, teas, aqueous extracts, alcohol extracts, candy, and gum.

13. The method of claim 1, wherein the nucleic acid sequence is deoxyribonucleic acid.

14. The method of claim 1, wherein the nucleic acid sequence is amplified using PCR.

15. The method of claim 1, wherein the nucleic acid sequence is amplified using real time PCR.

16. The method of claim 1, wherein the method further comprises distinguishing among species of Panax.

17. A method for distinguishing among species of Panax, the method comprising:
a. amplifying a polymorphic region of DNA from a plant using amplification primers comprising SEQ ID NOs:4-10, the primers producing a specific amplification profile of products in each species; and
b. identifying the plant by comparing its amplification profile against known amplification profiles of Panax species.

18. The method of claim 17, wherein the primers are at least seventy-five percent identical in nucleotide sequence to SEQ ID NOs:4-10.

19. A single stranded oligonucleotide consisting essentially of a nucleotide sequence selected from the group consisting of SEQ ID NOs:1, 2, 3, 4, 5, 6, 7, 8, 9, and 10.

20. The oligonucleotide of claim 19, wherein the oligonucleotide is at least seventy-five percent identical in nucleotides sequence to SEQ ID NOs:1, 2, 3, 4, 5, 6, 7, 8, 9, or 10.

21. An isolated nucleic acid selected from the group consisting of SEQ ID NOs:1, 2, 3, 4, 5, 6, 7, 8, 9, and 10.

22. The nucleic acid of claim 21, wherein the nucleic acid is at least seventy-five percent identical in nucleotide sequence to SEQ ID NOs:1, 2, 3, 4, 5, 6, 7, 8, 9, or 10.

23. A kit for determining whether a plant is a Panax species or an imposter species, the kit comprising:
a. amplification primers that produce a specific amplification profile of products in each species;
b. instructions for amplifying a polymorphic region of deoxyribonucleic acid from the plant; and
c. amplification profiles from Panax and imposter species for identifying the plant by comparing its amplification profile with the standard profiles.

24. The kit of claim 23, wherein the amplification primers comprise SEQ ID NOs:1-6.

25. The kit of claim 24, wherein the primers are at least seventy-five percent identical in nucleotide sequence to SEQ ID NOs:1-6.

26. The kit of claim 23, wherein the amplification primers comprise SEQ ID NOs:4-10.

27. The kit of claim 26, wherein the primers are at least seventy-five percent identical in nucleotide sequence to SEQ ID NOs:4-10.

28. The kit of claim 23, wherein the nucleotides comprising the amplification primers are selected from the group consisting of standard, nonstandard, modified, and derivatized nucleotides.

29. The kit of claim 23, wherein the Panax species is selected from the group consisting of P. quinquefolius, P. ginseng, P. notoginseng, P. bipinnatifidus, P. japonicus, P. pseudoginseng, P. trifolius, P. vietnamensis, P. wangianus, and P. zingiberensis.

30. The kit of claim 23, wherein the imposter species is selected from the group consisting of Eleutherococcus senticosis, Angelica sinensis, Aralia spp., Campanumoea pilosula, Caulophyllum thalictroides, Eleutherococcus gracilstylus, Eurycoma longifolia, Gynostemma pentaphyllum, Lepidium meyenii, Oplopanax horridus, Pfaffia paniculata, Pseudostellaria heterophylla, Rumex hymenosepalus, Trichopus zeylanicus, and Withania somnifera.

31. The kit of claim 23 wherein the Panax species is selected from the group consisting of P. quinquefolius, P. ginseng, and P. notoginseng, and the imposter species is Eleutherococcus senticosis.

32. The kit of claim 23, wherein the plant is a material selected from the group consisting of roots, root prongs, root fibers, stems, leaves, leaflets, flowers, and seeds.

33. The kit of claim 32, wherein the plant material is selected from the group consisting of whole, shredded, sliced, diced, crushed, pulverized, and heated material.

34. The kit of claim 23, wherein the plant is a processed plant material selected from the group consisting of capsules, tablets, powders, teas, aqueous extracts, alcohol extracts, candy, and gum.

35. The kit of claim 23, wherein the deoxyribonucleic acid is amplified using PCR.

36. The kit of claim 23, wherein the deoxyribonucleic acid is amplified using real time PCR.

37. The kit of claim 23, wherein the kit further comprises a reaction buffer solution, a dNTP mixture, a divalent cation solution, and Taq DNA polymerase.

38. The kit of claim 23, wherein the kit further comprises distinguishing among species of Panax.


SEQUENCE LISTING (10 sequences)

SEQ ID NO: 1 - DNA, 30 bp, Artificial
  cDNA24-F PRIMER
gccactatatttcccctatttctctcctcc

SEQ ID NO: 2 - DNA, 27 bp, Artificial
  cDNA24-R PRIMER
cagccatttagccttcgacttatgtgc

SEQ ID NO: 3 - DNA, 26 bp, Artificial
  TG24-R PRIMER
tttggttttgccttcttcaggtaata

SEQ ID NO: 4 - DNA, 24 bp, Artificial
  GITS-F PRIMER
tgaacctgcggaaggatcattgtc

SEQ ID NO: 5 - DNA, 20 bp, Artificial
  GITS-R PRIMER
actcgcatttgggccaaccg

SEQ ID NO: 6 - DNA, 25 bp, Artificial
  SGITS-R PRIMER
cgaggacggcacaacagggtcacat

SEQ ID NO: 7 - DNA, 30 bp, Artificial
  cDNA21-F PRIMER
tcagtactgggattacctgaatgctcttgg

SEQ ID NO: 8 - DNA, 26 bp, Artificial
  cDNA21-R PRIMER
cgagccatttggatcatgaaaggaag

SEQ ID NO: 9 - DNA, 28 bp, Artificial
  cDNA41-F PRIMER
actggctagctagctagctcgatcgctc

SEQ ID NO: 10 - DNA, 25 bp, Artificial
  cDNA41-R PRIMER
ggctctccctttgttgcattgaagt